(19) United States
(12) Patent Application Publication
(10) Pub. No.: US 2016/0331863 A1
(43) Pub. Date: Nov. 17, 2016

(54) METHODS FOR THE PREVENTION AND TREATMENT OF SKIN PROBLEMS EXACERBATED BY MOISTURE AND/OR FRICTION

(71) Applicant: Crawford Healthcare Inc., Knutsford (GB)

(72) Inventor: Garrett D. Cawthon, Frankfort, KY (US)

(73) Assignee: Crawford Wondcare Ltd.

(21) Appl. No.: 14/947,875

(22) Filed: Nov. 20, 2015

Related U.S. Application Data
(63) Continuation of application No. 14/133,557, filed on Dec. 18, 2013, now abandoned.

(51) Int. Cl. A61L 26/00; A61M 35/00; A61M 11/00

(52) U.S. Cl. CPC A61L 26/0061; A61L 26/0076; A61L 26/0004; A61L 26/0057; A61M 11/007; A61M 35/003; A61L 2300/802

(57) ABSTRACT

A method for treating skin problems exacerbated by moisture and/or friction includes identifying a skin treatment area having a wound which produces exudates or other adverse skin condition, determining that the skin treatment area is at risk for at least one of skin maceration, friction and shearing and, in response to the determining, applying a thin layer of a composition to the skin treatment area. An exemplary composition comprises from about 1 percent to about 40 percent by weight of a solid particulate material such as zinc oxide, up to about 30 percent by weight of water, and from about 20 percent to about 99 percent by weight of a fluid base material selected from the group consisting of mineral oil, silicone oil, plant-based oil and mixtures thereof. The applying includes spraying the composition on the skin treatment area with an atomizer.

### BACKGROUND

A variety of problematic skin conditions afflict the human body, these conditions being caused by a wide variety of external factors, such as injury, moisture, friction, extreme temperature, chemical irritants, insect bites, and microbiological infection. Problems can also originate intrinsically with various dermatological diseases such as eczema and hives.

Moisture and friction are two of the most common factors that inhibit healing of such skin conditions in that they foster further degradation of the skin or wound area. Maceration is the weakening of the skin that results from prolonged contact with moisture, rendering the skin susceptible to damage from friction or shear. These factors often work together to make skin breakdown even more prevalent and problematic.

Various topical treatments, such as ointments, creams, and lotions, have been used to treat such skin conditions. Most of these products are based on protecting the skin using a skin protectant at a designated concentration, as listed in the FDA monograph for Skin Protectants. Proper application is required to maximize the effectiveness of the treatment products. For example, many products require a high frequency of application—in some cases three to six times per day—to maintain adequate treatment. Compliance to this treatment protocol is often difficult for both caregiver and patient.

Another disadvantage associated with ointments, creams, and lotions is the caregivers' consideration that “more is better” when dispensing. Supported by the manufacturers' claims and/or direction, caregivers often lather a thick coating onto the skin. One result is a significant amount of wasted material during the application of rubbed-in products.

Many ointments, creams, and lotions are highly diluted with water, and contain significant amounts of other ingredients (e.g., preservatives, pH modifiers, and surfactants) that are necessary to integrate hydrophobic ingredients into the water-based solution or impart other desired features. When the water evaporates, these ingredients increase in concentration, which can cause irritation to the skin. For example, a 0.5% methylparaben preservative is not irritating in an 80% water solution but, after the water has evaporated, the remaining preservative concentration increases to 2.5%, a level that can readily cause irritation.

Thick coatings of ointments, creams, and lotions are especially problematic in that they inhibit oxygen from passing through the coating to the skin/wound site. Oxygen is needed to promote cell growth, and starving the wound of oxygen inhibits healing. Moreover, while thicker coatings protect from external moisture, they also have the additional drawback of inhibiting internal moisture, such as exudate and perspiration, from leaving the skin surface. Containment of such exudate and/or perspiration in contact with the skin can result in skin maceration that weakens the skin and makes it more susceptible to infection, skin tears, friction and the like. Moreover, the moistened, warm environment promotes microbial and fungal growth that can lead to infection.

Another problem with typical prior art ointments, creams, and lotions is that they become dry, gritty, sticky, and/or irritating to the skin as the water evaporates from formulation. Clean-up is difficult because the dry materials must be physically removed from the skin's surface using aggressive pressure. This removal can break down the healing skin and create pain to the patient. Moreover, inadequate and difficult removal of residues prevents the use of such materials in deeper, more problematic wounds, such as, for example, venous stasis ulcers or Stage II-IV pressure ulcers.

There remains a need for improved topical products for treating skin conditions that address one or more of the above and/or other problems and/or improve outcomes compared to products in the prior art. The present invention addresses this need and provides other benefits and advantages.

### SUMMARY

In general, the present invention is directed to compositions and methods for treating and preventing skin problems that are exacerbated by moisture and friction. The compositions described herein, applied as described herein, lubricate and/or moisturize the skin by providing a thin layer—for example, less than half a millimeter—of the composition over the skin treatment area. While not intending to be bound by any theory, it is believed that a thin coating of one of the disclosed compositions is effective in part because the water content is considerably below that of other comparable products, providing a hydrophobic formula that strongly and immediately adheres to the skin to create a moisture barrier.

Hydrophobic treatment compositions disclosed herein promote healing of problem skin areas. The compositions have antimicrobial and antifungal properties, are nontoxic, and form a moisture barrier for external moisture while allowing internal moisture (e.g., exudate and perspiration) to diffuse through the coating to prevent maceration. The compositions also allow oxygen to diffuse to the skin through the coating to promote healing. This disclosure also describes the spray application of the compositions to a selected skin treatment area without the need for the person administering the treatment to directly contact the composition or the skin. Application of the treatment composition is accomplished by forming the composition into a mist or spray using an atomizing spray dispenser.

While the actual nature of the invention covered herein can only be determined with reference to the claims appended hereto, certain forms of the invention that are characteristic of the embodiments disclosed herein are described briefly as follows.

In one form of the invention, a method includes selecting a skin treatment area having an injury, wound, or other adverse skin condition, and applying a treatment composition onto the skin treatment area. In certain embodiments, the selecting includes identifying a skin location having an open or weeping wound or that is at risk for skin maceration.

The composition includes a fluid base material, and in certain embodiments further includes a solid particulate material. In some embodiments, the solid particulate material is zinc oxide, and the fluid base material comprises at least one of a mineral oil, a plant-based oil, and a silicone oil.

The composition may further include one or more additives, such as for soothing the wound, providing a selective moisture barrier, fighting infections, modifying the viscosity, or altering the appearance or scent of the composition.

Applying the composition is preferably performed such that only the composition comes into contact with the treatment area. In other words, the wound is not contacted by an application medium, such as a swab, pad, or finger. This is accomplished by passing the composition through a spraying mechanism, such as an atomizer or mister.

The compositions and application methods disclosed herein have a variety of advantages over traditional rub-in ointments, creams and lotions in the prior art, including for example one or more of the following: (i) in view of the concentrated, strongly hydrophobic characteristics of various composition embodiments described herein, less material is required for adequate treatment/protection; (ii) spray application substantially reduces the likelihood of skin breakdown that is more likely to occur when using a rub-in ointment; (iii) composition embodiments that are clear/translucent allow viewing of the skin wound bed versus opaqueness associated with most traditional ointments, creams and lotions; (iv) spray application can be achieved quickly and the applied compositions are resistant to rub-off and run-off; (v) low viscosity of the compositions under shear provides improved wetting of the skin and helps by-pass physical barriers such as hair; (vi) spray application reduces waste of the product; (vii) spray application reduces or eliminates physical contact by the caregiver, which can be painful; (viii) skin pain is reduced when treating wounds with open skin and/or exposed nerve endings by the oily ingredients coating the open skin/nerve endings; (ix) oxygen is allowed to transfer to the skin/wound site at a higher rate; (x) moisture (e.g., exudates and perspiration) is allowed to transfer from the skin/wound site at a higher rate to prevent maceration of the skin; (xi) drying of the skin is avoided in embodiments with high oil content and low water content; (xii) highly hygroscopic composition embodiments adhere to the skin and are difficult to rub-off or wash-off; (xiii) highly hygroscopic composition embodiments dehydrate and kill bacteria and fungi on the skin; (xiv) highly hydrophobic composition embodiments protect the skin from external moisture.

Further embodiments, forms, features , aspects, benefits, objects and advantages of the present invention will become apparent from the detailed description and figures provided herewith.

### BRIEF DESCRIPTION OF DRAWINGS

FIG. 1 is a side elevational view, with a cut-out portion, of an exemplary atomizing spray dispenser.

FIG. 2 is a sectional side elevational view of a representative atomizing pump spray dispenser.

FIG. 3 is a sectional side elevational view of a representative piston-style dispenser.

FIG. 4 is a sectional side elevational view of a first representative bag-in-can style dispenser.

FIG. 5 is a sectional side elevational view of a second representative bag-in-can style dispenser.

FIG. 6 is a sectional side elevational view of a representative aerosol dispenser.

### DETAILED DESCRIPTION

For the purposes of clearly, concisely and exactly describing exemplary embodiments of the invention, the manner and process of making and using the same, and to enable the practice, making and use of the same, reference will now be made to certain exemplary embodiments and the figures, and specific language will be used to describe the same. It shall be understood that no limitation of the scope of the invention is thereby created, and that the invention includes and protects such alterations, modifications, and further applications of the exemplary embodiments as would occur to one skilled in the art to which the invention relates.

The present disclosure involves treatment compositions and methods that promote healing to adverse skin conditions, such as those associated with friction and moisture. The compositions described herein are both antimicrobial and antifungal. Without being bound to any theory, it is believed that the antimicrobial and antifungal properties of the compositions result at least in part from their hygroscopic properties, by which they remove water from the skin surface at the wound site, which water is necessary for microorganisms and fungi to live and grow. In addition, the compositions form a hydrophobic moisture barrier to protect the skin from external moisture (e.g., urine, feces, water) while allowing internal moisture (e.g., exudate and perspiration) to diffuse through the coating to prevent maceration. Moreover, oxygen can transfer back through the coating to the skin to promote cell growth and healing. Due to the high level of hydrophobic fluids (e.g., mineral oil, silicone oil, plant-based oils or mixtures thereof), the compositions do not become dried and caked on the skin, even if remaining on the skin for an extended period of time, minimizing friction and irritation while continuously lubricating the skin over an extended period of time. In addition, the strong adherence of the formula to skin minimizes the amount of the composition needed for therapeutic coverage and reduces waste during application.

The compositions and methods disclosed herein address a number of problems associated with wounds exacerbated by moisture and/or friction and other adverse skin conditions by providing methods, compositions and systems for treatment involving application of a treatment composition as an atomized spray. Described herein are methods for applying a variety of treatment compositions to a selected treatment area without the need for the person administering the treatment to directly contact the composition or the skin of the patient.

Generally speaking, the method includes selecting a skin treatment area having an injury, wound, or other adverse skin condition, and in response to the selecting, applying a treatment composition onto the skin treatment area. Illustrative embodiments of the composition and the delivery mechanism will be first described, followed by processes for selecting the skin treatment area.

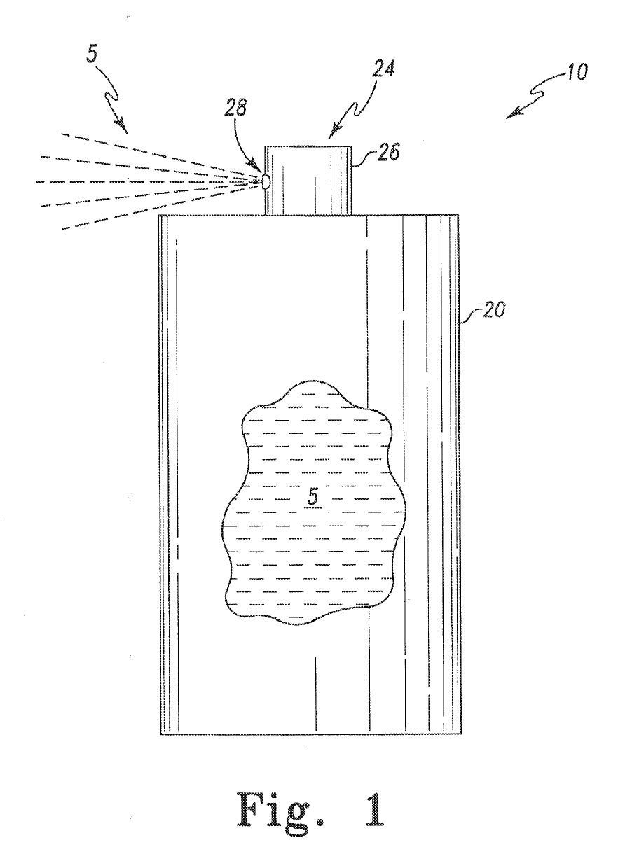

Referring to FIG. 1, to practice the method, a treatment composition 5 is placed into an atomizing spray dispenser 10. The dispenser is preferably a container 20 equipped with a spray delivery mechanism 24 configured to atomize and propel the composition toward a treatment surface. Compositions described herein exhibit excellent features for the treatment of skin injured by moisture and/or friction. When a proper amount of the composition is atomized and propelled by a suitable atomizing spray delivery mechanism, the composition forms a coating over the skin surface, thereby providing a barrier to moisture, and a soothing function to the skin.

It is preferred that the composition have a suitably low viscosity that it may be readily converted to an atomized spray, but that the viscosity not be too low because a viscosity that is too low may result in the composition running off of the treatment surface. Compositions disclosed herein have non-Newtonian, i.e., thixotropic, properties. The composition may comprise a liquid, a combination of liquids or a combination of one or more liquids and one or more solids that features a suitable viscosity. Proper viscosity characteristics can be attained at least in part by the selection of a fluid base material.

The term “fluid base material” is used herein to refer to a fluid into which additional ingredients may be mixed, dissolved and/or suspended. In one embodiment, the fluid base material comprises an oil having a viscosity of from about 1 centipoise to about 1000 centipoise. Exemplary oils that may be used are mineral oils, silicone oils, plant-based oils or mixtures thereof. The term “mineral oil” is used herein to refer to a liquid petroleum derivative. Exemplary commercially available mineral oils that may be used include Witco Corporation's CARNATION™ Mineral Oil (having a viscosity range of 9-12 centipoise) or Penreco Corporation's DRAKEOL™ 7 Mineral Oil (having a viscosity of 9-11 centipoise).

The term “silicone oil” is used herein to refer to a liquid silicone composition. Exemplary silicone oils that may be used, for example, are cyclomethicone, dimethicone and derivatives thereof. Commercially available silicone oils include Dow Corning Corporation's 200 polydimethylsiloxane fluids and GE Silicones' SF series of low viscosity dimethyl fluids. Cyclomethicone is a volatile compound, and is advantageously included in certain inventive compositions because it evaporates when applied to the skin's surface, making the coating more dry to the touch. Other volatile compounds may also be used in this way, including, for example, an organic solvent such as isododecane. Such compounds are particularly useful in embodiments that utilize an aerosol dispenser as the delivery mechanism (described in more detail below), because the volatile compounds may be used as liquid solvents in the container that evaporate when dispensed from the container, leaving a drier coating on the skin surface.

The term “plant-based oil” is used herein to refer to a non-petroleum biomass derived oil, such as a vegetable or fruit oil. By way of non-limiting example, the plant-based oil may include, for example, at least one of linseed oil, jojoba oil, rapeseed oil, soybean oil, grape seed oil, and sunflower oil. It is of course not intended that the fluid base material be limited to the above exemplary compositions. As an alternative to plant-based oil, silicone oil and mineral oil, another material that may be used as the fluid base material, or included therein, is cod liver oil.

The fluid base material may alternatively be water, an oil-in-water emulsion or a water-in-oil emulsion. Inventive compositions including water or an emulsion as the base material may be preferable for some uses, as such compositions have a less greasy feel. The inclusion of water in the composition can provide additional advantages, such as reducing the cost of the composition, providing a source of moisture for dry skin, and enabling inclusion of additional hydrophilic ingredients that would not become suitably dissolved or dispersed in an oil-based composition.

When water is included in a composition that also comprises one or more oils, it is readily understood that an emulsifying agent will typically be necessary to produce a stable mix of the water and nonaqueous ingredients in the composition. Examples of suitable emulsifiers that may be used include cetyl alcohol, stearyl alcohol, cetearyl alcohol and glycerol monolaurate. It is of course not intended that this list be limiting but simply provide examples of emulsifiers that may be used. Additional suitable emulsifiers are set forth on pages 90 to 94 of the C.T.F.A. Cosmetic Ingredient Handbook, First Edition, 1988, which is hereby incorporated by reference. Further guidance to the preparation of stable emulsions generally may be found in U.S. Pat. No. 5,417,961 to Nearn et al., which is hereby incorporated by reference in its entirety.

In certain embodiments, the composition comprises at least about 1 percent solid particulate material by weight and at least about 20 percent by weight of a fluid base material. The solid particulate material may include one or more of particulate zinc oxide, talc, calamine or kaolin. Each of these particulate materials is hygroscopic—that is, they tend to absorb water. The hydrophobic oil-based fluid restricts the diffusion of water to the hygroscopic solid, resulting in a controlled absorption of moisture, so as to not over-dry the skin's surface.

The hygroscopic components help absorb moisture—such as exudate and perspiration that diffuses through the skin surface. Other absorptive compounds, for example, humectants, could be used for this purpose, but the preferred materials (zinc oxide and petrolatum, discussed below) are also FDA-approved active ingredients as skin protectants. Thus, compositions described herein can have the recommended concentrations of skin protectants while minimizing additional ingredients that could be irritating, or have other side-effects.

In one embodiment, a composition as described herein is applied to a skin treatment area to form a coating having a thickness no greater than 0.5 mm. In alternative embodiments, the coating has a thickness no greater than 0.4 mm, no greater than 0.3 mm, no greater than 0.2 mm or no greater than 0.1 mm. In another embodiment the composition applied to the skin treatment area to form a coating is applied in an amount of from about 0.1 grams per 100 cm2 to about 3 grams per 100 cm2. In alternative embodiments, the composition is applied in an amount of from about 0.1 grams per 100 cm² to about 2 grams per 100 cm², from about 0.1 grams per 100 cm² to about 1.5 grams per 100 cm², from about 0.1 grams per 100 cm² to about 1.5 grams per 100 cm² or from about 0.2 grams per 100 cm² to about 1.5 grams per 100 cm².

The low water fraction in the hydrophobic formulas reduces the amount of caking on the skin as the water evaporates. This is in contrast to the hard, gritty residue resulting from the drying of ointments, lotions, and creams. Such residue irritates the skin, and creates additional friction for skin-to-skin and skin-to-cloth contact areas. The compositions used in the disclosed methods may retain liquidity on the skin. The liquidity of the composition enables it to continue lubricating the skin, thereby minimizing friction and shear, and may also enable the composition to continue to knead the therapeutic ingredients into the skin's cracks and crevices.

One advantage of the compositions and methods described herein is that moisture, either as perspiration or exudate, can readily leave the skin through the coating; the skin remains dry because the moisture cannot re-adhere to the skin due to the highly hydrophobic nature of the coating. The body's internal pressure that forces the liquid through the skin is sufficient to overcome the barriers associated with the hydrophobicity and diffusional resistance associated with the coating. This is in contrast to the thicker coating associated with ointments that trap and accumulate moisture intimately on the skin surface, leaving the skin vulnerable to maceration.

In embodiments in which the particulate material include zinc oxide, the zinc oxide can have an average particle size of from about 0.01 microns to about 100 microns. In another embodiment, the average particle size is from about 0.01 microns to about 10 microns and in yet another embodiment from about 0.01 microns to about 1 micron. Zinc oxide particles of from about 0.01 microns to about 0.1 microns are commonly referred to as microfine zinc oxide particles. A feature of microfine zinc oxide that is desirable in some applications is that the microfine zinc oxide is translucent when dispersed in a conventional fluid base material. Compositions including microfine zinc oxide therefore do not have the characteristic opaque white appearance of formulations including zinc oxide of larger particle sizes. It is also believed that microfine zinc oxide provides additional advantages over larger particulates, including a smoother film. coating on the skin, lower viscosity at the same overall zinc oxide loading, better penetration of the zinc oxide into the skin's cracks and crevices, more even coverage, and higher surface area providing for more reaction and therapeutic activity.

It is also intended that the term “zinc oxide” encompass coated zinc oxides. A typical coating is silicone-based and is used to help disperse the zinc oxide in solution and to lower the viscosity of a zinc oxide mixture. It has been reported that uncoated zinc oxide particles swell in certain solvents, such as, for example, octyl palmitate. One advantage of using coated zinc oxide is that silicone coated zinc oxide particles are hydrophobic, non-reactive and not affected by water. Coated hydrophobized zinc oxides or particles also have a significantly decreased photoreactivity, which makes them more resistant to degradation and more chemically inert than non-coated zinc oxides. Hydrophobized zinc oxide is therefore particularly useful in compositions that are applied to skin that is exposed to sunlight or other ultraviolet radiation.

The ratio of zinc oxide to fluid base material in this composition is preferably selected such that the composition has a predetermined viscosity at a given temperature. It is readily understood that the ratio selected is dependent upon the desired viscosity for a given system. For example, different delivery systems may function optimally when used to deliver compositions having different overall viscosities. Further, the preferred ratio also depends upon the viscosity of the fluid base material selected for use and upon the particle size distribution of the zinc oxide. These and other factors may be readily determined and considered by a person of ordinary skill in the art, without undue experimentation, to make an inventive composition having a suitable viscosity. The composition in one embodiment comprises from about 1 to about 40 percent zinc oxide by weight. In alternative embodiments, the zinc oxide content of the composition is from about 5 to about 25 percent or from about 10 to about 15 percent. In one embodiment, the composition also comprises from about 20 to about 99 percent fluid base material by weight. In alternative embodiments, the fluid base material content of the composition is from about 33 to about 80 percent or from about 55 to about 75 percent.

The composition may also optionally include one or more of a wide variety of beneficial additives that may be incorporated for a variety of reasons. For example, the composition may comprise a skin conditioning agent, for example lanolin or cod liver oil. It is understood that the term “lanolin” refers to the various forms of lanolin and its derivatives, such as, for example, lanolin oil, lanolin wax, and lanolin alcohols.

The composition may also include a moisture barrier material, such as paraffin, microcrystalline wax. Additionally or in the alternative, the moisture barrier material may include a hygroscopic petrolatum. Additional ingredients that may optionally be included in a composition are fragrances, dyes, preservatives, anti-bacterial agents, anti-fungal agents and emollients. It is of course not intended that this list limit the scope of the invention, but simply provide examples of ingredients that might be included in the compositions.

When including one or more beneficial additives, it is important to consider the effect that the one or more additives have on the overall viscosity of the composition. Thus, adjustments of the zinc oxide to fluid base material ratio may be necessary to achieve a proper viscosity when including one or more additives to achieve a desired combination of additional features.

In certain exemplary embodiments, the composition may also include from about 1 to about 16 percent lanolin by weight. In other alternative embodiments, the lanolin content of the composition is from about 5 to about 15 percent or from about 5 to about 10 percent. The composition may further include from about 1 to about 25 percent petrolatum by weight. In other alternative embodiments, the petrolatum content of the composition is from about 5 to about 15 percent or from about 5 to about 10 percent. The composition may further include from about 1 to about 12 percent cod liver oil by weight. In other alternative embodiments, the cod liver oil content of the composition is from about 5 to about 12 percent or from about 5 to about 10 percent.

An exemplary composition in the practice of the invention comprises from about 1 to about 40 percent zinc oxide, from about 20 to about 99 percent fluid base material, from about 0 to about 16 percent lanolin, from about 0 to about 12 percent cod liver oil and from about 0 to about 25 percent petrolatum, all by weight. In one embodiment, the fluid base material comprises from about 20 to about 100 percent mineral oil and from about 0 to about 20 percent silicone oil by weight. Alternatively, the fluid base material may comprise from about 20 to about 100 percent silicone oil and from about 0 to about 20 percent mineral oil by weight.

Another composition embodiment comprises from about 5 to about 25 percent zinc oxide, from about 33 to about 80 percent fluid base material, from about 5 to about 15 percent lanolin, from about 5 to about 12 percent cod liver oil and from about 5 to about 15 percent petrolatum, all by weight. An additional composition embodiment comprises from about 10 to about 15 percent zinc oxide, from about 55 to about 75 percent fluid base material, from about 5 to about 10 percent lanolin, from about 5 to about 10 percent cod liver oil and from about 5 to about 10 percent petrolatum, all by weight. The composition may also advantageously include from. about 5 to about 10 percent talc and/or microcrystalline wax to increase the viscosity of the composition. In one embodiment, the composition is clear or translucent.

A composition that has been found to have particularly excellent features in accordance with the invention comprises about 25 percent zinc oxide, about 5 percent lanolin, about 5 percent petrolatum, about 5 percent microcrystalline wax, about 10 percent cod liver oil, about 40 percent mineral oil and about 10 percent cyclomethicone, all by weight.

In an alternate aspect of the invention, there is provided a treatment composition that comprises a fluid base material and a source of vitamin A (also referred to as “retinol”) and vitamin D (also referred to as “cholecalciferol”). An excellent source of vitamins A and D in this aspect of the invention is cod liver oil; however, it is understood that alternate sources may be used as alternatives, or in addition to, cod liver oil. For example, the vitamin source may comprise purified vitamin A, purified vitamin D and/or vitamin .A palmitate. In one embodiment, the composition comprises lanolin, petrolatum, cod liver oil and a fluid base material (which may also include cod liver oil). For example, the composition may comprise from about 1 to about 16 percent lanolin, from about 47 to about 97 percent fluid base material, from about 1 to about 12 percent cod liver oil and from about 1 to about 25 percent petrolatum, all by weight. Another composition embodiment comprises from about 5 to about 16 percent lanolin, from about 57 to about 85 percent fluid base material, from about 5 to about 12 percent cod liver oil and from about 5 to about 15 percent petrolatum, all by weight. An additional composition embodiment comprises from about 10 to about 16 percent lanolin, from about 57 to about 70 percent fluid base material, from about 10 to about 12 percent cod liver oil and from about 10 to about 15 percent petrolatum, all by weight. It is understood that, where the fluid base material wholly or partially comprises cod liver oil, the cod liver oil is intended to satisfy the recitation of the cod liver oil and the fluid base material.

A composition that has been found to have particularly excellent features in accordance with the invention comprises about 10 percent cod liver oil, about 10 percent lanolin, about 10 percent petrolatum, about 8 percent microcrystalline wax, about 47 percent mineral oil, and about 15 percent cyclomethicone, all by weight.

As mentioned above, other additives may be included in various alternative composition embodiments, including conventional additives typically employed in skin care compositions. For example, fragrance oils may be used to mask the odor of other ingredients and/or for cosmetic appeal. Dyes may also be used to color the composition. In addition, preservatives, such as, for example, DMDM hydantoin, methylparaben or other esters of parahydroxy benzoic acid, and the ester of propylparahydroxy benzoic acid and benzethonium chloride may be used. Other emollients such as aloe vera and vitamins A, D and E may also be used. Emulsion compositions described herein can also optionally include additional active ingredients such as antimicrobial agents, antibacterial agents and antifungal agents. In certain embodiments, silver is included as a broad-spectrum anti-microbial agent, for example in a concentration of 10-3000 parts per million.

In one embodiment, the composition has a viscosity of from about 1 to about 1000 centipoise, measured at room temperature using a Brookfield viscometer. In another embodiment, the viscosity is from about 200 to about 700 centipoise. In yet another embodiment, the viscosity is from about 300 to about 500 cemtipoise.

In one embodiment, the composition comprises from about 1 percent to about 40 percent by weight of a solid particulate material such as zinc oxide, from about 20 percent to about 99 percent by weight of a fluid base material such as mineral oil, silicone oil, plant-based oil and mixtures thereof, and from 0 to about 30 percent by weight of water. In these and other embodiments, the composition may further comprise one or more member selected from the group consisting of talc, lanolin, cod liver oil, petrolatum, paraffin wax and microcrystalline wax. In these and other embodiments, the composition may include at least one of a fragrance, a dye, a preservative, and an emollient.

In another illustrative embodiment, the composition comprises from about 20 percent to about 30 percent by weight zinc oxide, from about 30 percent to about 60 percent by weight of a fluid base material comprising mineral oil and cyclomethicone, from about 1 percent to about 16 percent by weight of lanolin, from about 1 percent to about 25 percent by weight of petrolatum, from about 1 percent to about 10 percent by weight of microcrystalline wax, and from about 5 percent to about 12 percent by weight of cod liver oil.

In certain embodiments, the composition comprises from about 5 percent to about 30 percent zinc oxide by weight; and from about 20 percent to about 95 percent by weight of a fluid base material comprising at least one of mineral oil, silicone oil, and plant-based oil.

In an exemplary embodiment, the composition comprises from about 10 percent to about 15 percent zinc oxide by weight, from about 1 percent to about 16 percent by weight lanolin, from about 1 percent to about 12 percent by weight cod liver oil, from about 1 percent to about 25 percent by weight petrolatum, up to about 30 percent by weight water, and from about 47 percent to about 97 percent by weight of a fluid base material comprising at least one of mineral oil, silicone oil, and plant-based oil.

In another exemplary embodiment, the composition comprises from about 10 percent to about 30 percent by weight of hydrophobized zinc oxide, and from about 20 percent to about 90 percent by weight of a fluid base material selected from the group consisting of mineral oil, silicone oil, plant-based oil and mixtures thereof.

In certain embodiments, the composition comprises particulate zinc oxide, at least one of an anti-bacterial and an anti-fungal material, and at least one of a mineral oil, a silicone oil, a plant-based oil, and cod liver oil.

A composition as described herein may be made by mixing the ingredients and heating the mixture to an elevated temperature below the decomposition temperature of the ingredients. Heating is useful to ensure that any solid ingredients are melted, dissolved and/or dispersed in the composition and to more efficiently and stably achieve an acceptable degree of mixing. The mixture is preferably heated to a temperature in a range of from about 40° C. to about 75° C. When temperature sensitive ingredients, such as, for example, herbal ingredients and cod liver oil, are included in an inventive composition, these ingredients should be added after the mixture's temperature has been lowered to below about 40° C. When cod liver oil is used as the fluid base material, it is understood that the mixing should be conducted at a temperature below the decomposition temperature of the cod liver oil. A high shear mixer, such as a triple roll mixer or Charles Ross & Son Company's high-speed mixer-emulsifier, may advantageously be used to assist in the development of a uniform, stable composition.

Referring to FIG. 1, the composition can be applied to a skin treatment surface by atomizing the composition 5 and propelling the atomized composition toward the surface using a suitable atomizing spray dispenser 10 comprising a container 20 and an atomizing spray delivery mechanism 24. The illustrated atomizing spray delivery mechanism 24 releases the composition 5 from the container 20 through an outlet port 28 when a valve is mechanically actuated. In the illustrated embodiment, the mechanism 24 features a reciprocating actuator 26.

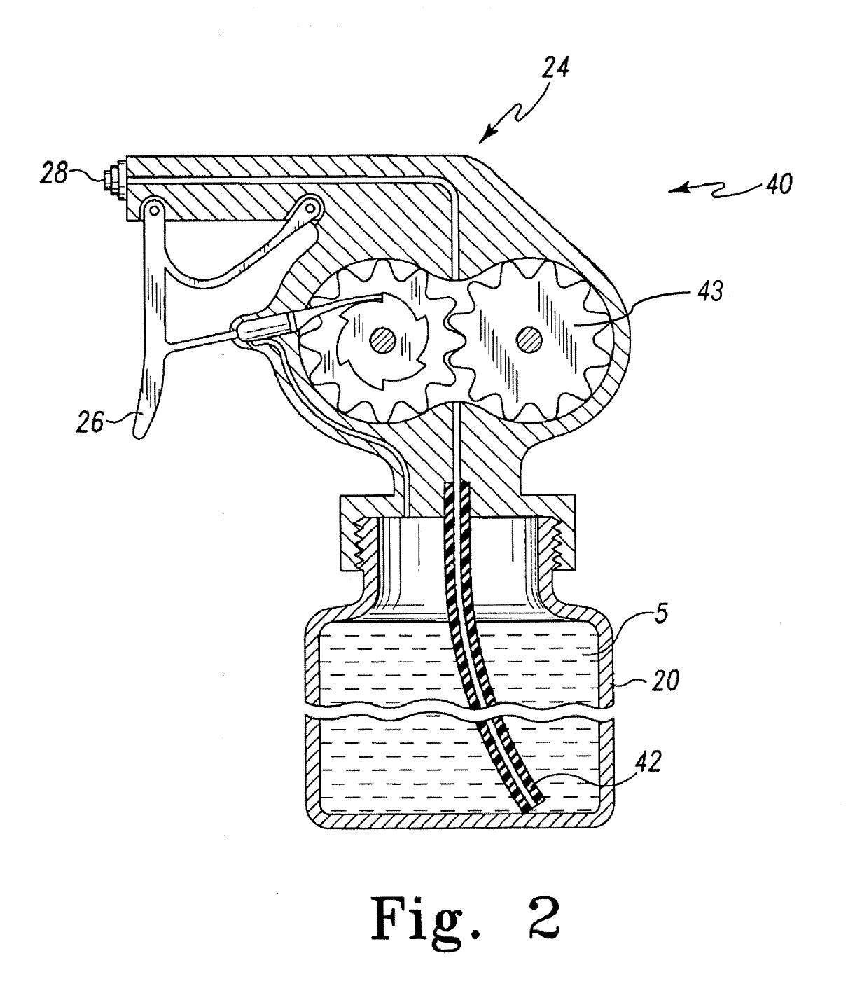

An exemplary atomizing spray delivery mechanism that may be used in accordance with the invention is an atomizing pump spray dispenser 40, a representative example of which is depicted in FIG. 2. As used herein, the term “atomizing pump spray dispenser” is intended to refer to a device that, upon activation of a mechanical pump, such as gear pump 43 of FIG. 2, draws a composition 5 from a container 20, atomizes the composition, and propels the atomized composition substantially in a predetermined direction. It is understood that the composition 5 drawn from container 20 preferably enters a conduit in fluid communication with the pump and having an inlet port 42 positioned near the bottom of container 20. “Atomizing” a composition refers to the separation of the composition into relatively small unitary masses (i.e., typically on the order of about 1 to about 100 microns).

It is important to recognize that the term “pump dispenser” has been used in the prior art to refer to devices for drawing a cream, lotion or ointment from a container and propelling a stream of the cream, lotion or ointment, such as, into the palm of ones hand. This type of dispenser is distinguished from an “atomizing pump spray dispenser” as described herein, because a pump dispenser for propelling a stream of a cream, lotion or ointment is incapable of atomizing the cream, lotion or ointment into an atomized mist or spray. It is understood that the unitary masses, when propelled from. an atomizing pump spray dispenser, form a mist or a spray. When the atomized composition is directed toward a given surface at a suitable velocity, the unitary masses adhere to the surface to provide a thin, often buttery, coating of the composition on the surface.

A wide variety of atomizing pump spray dispensers are commercially available, as are a wide variety of designs for actuating the dispensers and for atomizing the composition. While it is not intended that the invention be limited by the design of the atomizing pump spray dispensers, representative examples are set forth in U.S. Pat. No. 5,639,025 to Bush, U.S. Pat. No. 5,881,925 to Ando, and U.S. Pat. No. 5,249,747 to Hanson et al.

Another atomizing spray dispenser that may be used in accordance with the invention is a pressure release device. As used herein, the term “pressure release device” is intended to refer to a device that contains a composition under pressure, and, when actuated, opens a valve to release the composition from the pressurized compartment, atomizes the composition, and propels the atomized composition substantially in a predetermined direction by using energy provided by the force of the pressure. The composition may advantageously be maintained under pressure by placing the composition in a pressurized compartment of the container. The composition is releasably contained in the pressurized compartment and when a manually actuating valve is opened, the composition is released from the compartment, atomized, and released from the device as an atomized spray.

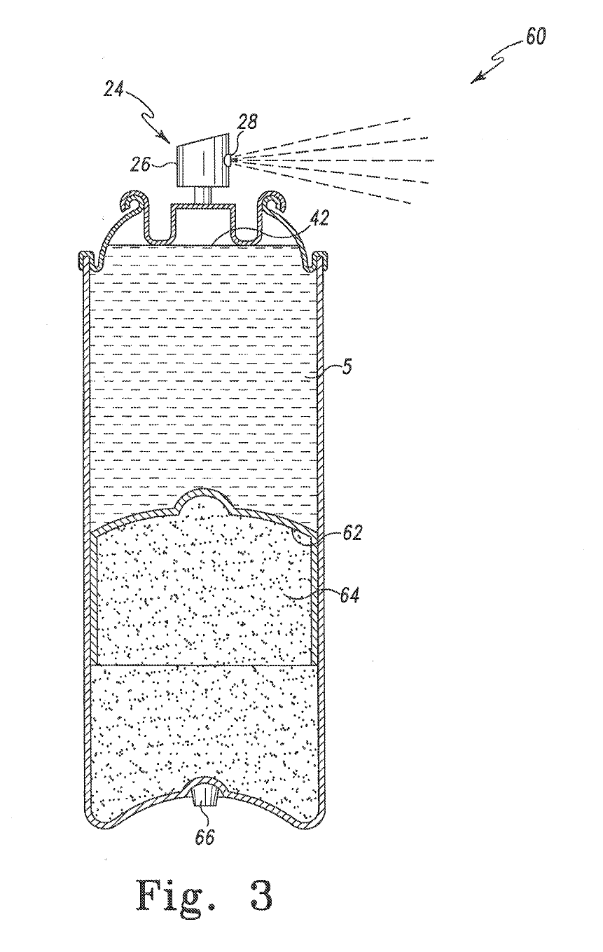

In certain embodiments, a representative example of which is depicted in FIG. 3, the pressure release device is a piston-style dispenser 60, and pressure is maintained on the composition 5 by pressure of a piston 62. In the illustrated embodiment, the pressure of the piston 62 is provided by placement of a pressurized gas 64 beneath the piston 62, which placement may be advantageously achieved by introducing gas 64 through a gas charging port 66. Alternatively, pressure on the piston may be achieved by a spring loaded mechanism beneath the piston. Such piston-style dispensers are commercially available, as are a wide variety of designs for actuating the dispensers and for atomizing the composition contained therein. While it is not intended that the invention be limited by the design of the piston-style can, representative examples are set forth in U.S. Pat. No. 4,134,523 to Hansen.

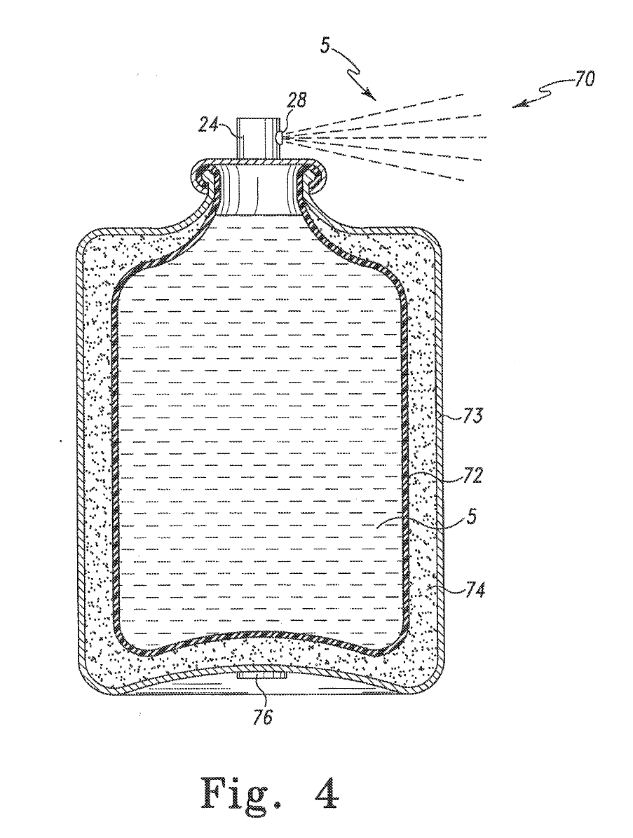

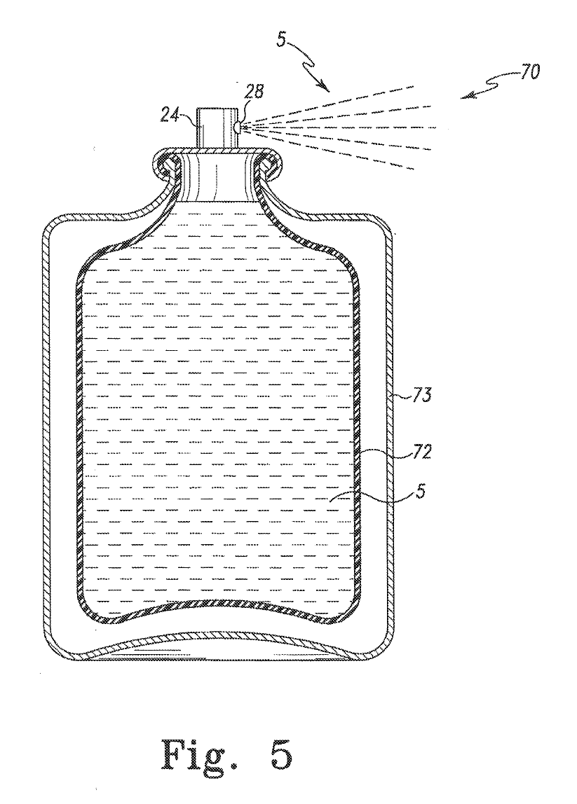

In other embodiments, representative examples of which are depicted in FIGS. 4 and 5, the pressure release device comprises a bag-in-can-style dispenser 70. The pressurized compartment of such a container is a polymeric bag 72 received inside a rigid can 73. Pressure is maintained on the composition 5 inside the bag 72. In one embodiment, a representative example of which is depicted in FIG. 4, the pressure is maintained upon the composition by a pressurizing gas 74 received in the can 73 and externally to the bag 72. In the illustrated embodiment, the pressurizing gas 74 can be placed in the can 73 by introducing gas 74 through a gas charging port 76. In another embodiment, an example of which is depicted in FIG. 5, the bag 72 is an elastic shape-memory bag, and pressure is maintained upon the composition 5 by maintaining the bag in an expanded state. This type of container is commonly referred to as a bladder pack container.

A wide variety of bag-in-can-style containers are commercially available, as are a wide variety of designs for actuating the dispensers and for atomizing the composition contained therein. While it is not intended that the invention be limited by the design of the bag-in-can-style container, representative examples are set forth in U.S. Pat. No. 3,788,521 to Laauwe, U.S. Pat. No. 4,510,734 to Banks et al., and U.S. Pat. No. 5,249,747 to Hanson et al.

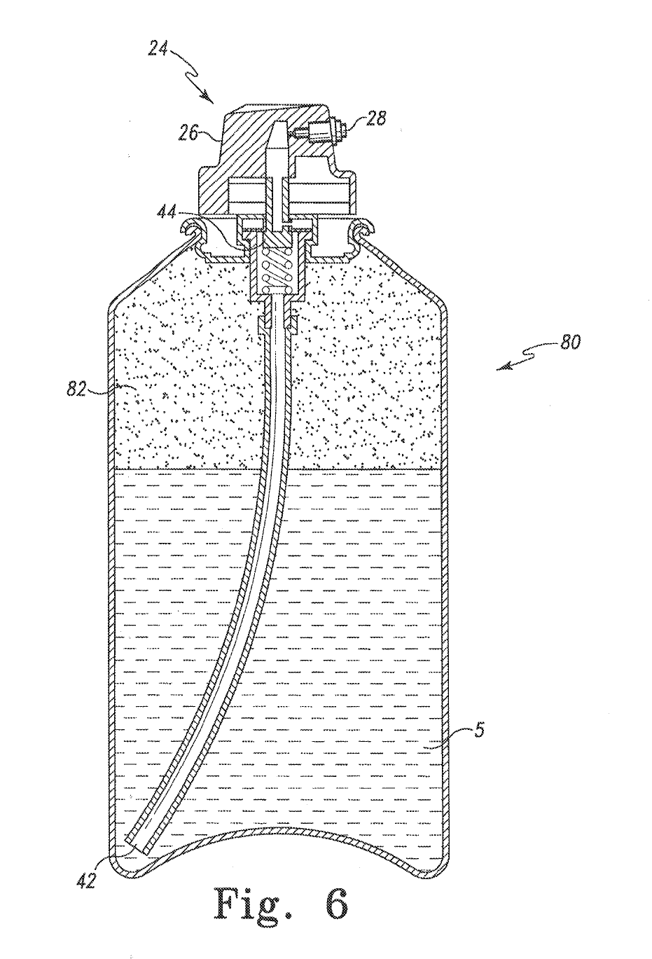

Another atomizing spray dispenser that may be used in accordance with the invention, a representative example of which is shown in FIG. 6, is an aerosol device 80. As used herein, the term “aerosol device” is intended to refer to a device that delivers the spray treatment composition by entraining the same in a carrier stream comprising an inert pressurized propellant gas 82. When such a device is actuated, the composition is released from the container, entrained in a gaseous stream, atomized, and propelled substantially in a predetermined direction by using energy provided by the pressurized gas. It is understood that the composition and carrier gas may alternately be contained in the container in a pre-mixed form, whereby actuation of the device results in a substantially constant release of the mixture. A wide variety of aerosol containers are commercially available, as are a wide variety of designs for actuating the dispensers and for atomizing the compositions contained therein. While it is not intended that the invention be limited by the design of the aerosol container, representative examples are set forth in U.S. Pat. No. 4,001,391 to Feinstone et al., U.S. Pat. No. 4,187,985 to Goth, U.S. Pat. No. 4,239,407 to Knight, U.S. Pat. No. 4,495,168 to Schmolka, U.S. Pat. No. 5,788,389 to de Laforcade, and U.S. Pat. No. RE030,093 to Burger.

While a wide variety of configurations and styles of atomizing spray dispensers are known in the art, the known prior art does not disclose or suggest the present invention, in which excellent treatment compositions are delivered to a skin surface from an atomizing spray dispenser. In view of the above, one system that may be used to achieve efficient treatment is depicted in FIG. 1 and includes a treatment composition as described herein and an atomizing spray dispenser 10 for atomizing the composition and delivering the atomized composition to a skin treatment area. In the illustrated embodiment, the dispenser 10 comprises a container 20, an atomizing spray delivery mechanism 24 affixed to the container 20, and the composition 5 positioned in the container 20. The mechanism includes an inlet port in fluid communication with the interior of the container for receiving the composition, a device for atomizing the composition, and an outlet port for propel ling the atomized composition substantially in a predetermined direction. It is readily understood that, in use, the inlet port must be in contact with the composition. In other embodiments, the spraying or atomizing may be achieved through the use of an atomizing pump spray dispenser, a piston-style dispenser, or an aerosol dispenser, as described above.

Having illustrated and described the composition and the delivery mechanism, exemplary methods for selecting a skin treatment area will now be described.

In certain embodiments, the selecting includes identifying a treatment area having a wound which produces exudate. In certain embodiments, the selecting includes identifying a skin treatment area having a weeping wound. In other embodiments, the selecting includes identifying a treatment area having a pressure ulcer. In some embodiments, the selecting includes identifying an open wound.

The selecting may further include determining a condition of the treatment area. For example, the selecting may include determining that the skin treatment area is at risk for maceration, and applying the composition based at least in part upon this determination. The selecting may further include determining that the skin treatment area is subject to at least one of friction and shearing, and the applying may be further based on this determination. The determination may be based, for example, on the treatment area being located in a skin fold.

The selecting may include determining that the treatment area will be exposed to ultraviolet radiation, for example due to exposure to the sun. In response to this determination, the composition applied to the treatment area may comprise a particulate matter including hydrophobized zinc oxide to prevent degradation of the composition or radiation damage to the underlying wound.

In certain embodiments, the selecting includes determining that the wound would benefit from both oxygen diffusion and a selective barrier to the passage of liquid. This may be based at least in part upon the nature of the wound. For example, open wounds and weeping wounds may require more oxygen diffusion than minor cuts and abrasions. In response to the determination, the composition is applied to the wound.

In some embodiments, the selecting includes determining that the skin treatment area is sensitive to the touch, for example due to exposed nerve endings. In response to the determination, the composition is sprayed onto the treatment area such that the treatment area does not come into contact with an applicator, thereby reducing the discomfort to the patient.

As will be appreciated by a person skilled in the art in view of the above descriptions, in one aspect of the present disclosure, there is provide a method that includes (i) identifying a skin treatment area having a wound which produces exudate; (ii) determining that the skin treatment area is at risk for at least one of skin maceration, friction and shearing; and (iii) based at least in part on the determining, applying a composition to the skin treatment area, the composition comprising from about 1 percent to about 40 percent by weight of a solid particulate material; from about 20 percent to about 99 percent by weight of a fluid base material comprising at least one material selected from the group consisting of mineral oil, silicone oil, plant-based oil; and from about 0 percent to about 30 percent by weight of water. The applying includes spraying the composition such that the sprayed composition contacts the skin treatment area.

In another aspect, the present disclosure provides a method that includes (i) identifying a skin treatment area having a wound or rash; (ii) determining that the skin treatment area would benefit from a covering that both allows oxygen diffusion and provides a selective barrier to the passage of liquid; (iii) providing a treatment system comprising a dispenser and a treatment composition; wherein the dispenser comprises a container and an atomizing spray delivery mechanism affixed to the container; and wherein the treatment composition is positioned in the container; (iv) passing the composition through the atomizing spray delivery mechanism to atomize the composition and to propel the atomized composition toward the skin treatment area; and (v) leaving said composition on the skin treatment area to form a coating. The treatment composition has a viscosity of from about 1 to about 1000 centipoise and comprises from about 1 percent to about 40 percent by weight of a solid particulate material, from about 20 percent to about 99 percent by weight of a fluid base material selected from the group consisting of mineral oil, silicone oil, plant-based oil and mixtures thereof, and from 0 percent to about 30 percent water by weight.

In yet another aspect of the present disclosure, there is provided a method that includes identifying a skin treatment area that is in need of antimicrobial treatment; and responding to the need by applying to the skin treatment area a composition as disclosed herein; wherein the applying includes passing the composition through an atomizing spray delivery mechanism and into contact with the skin treatment area to form a coating on the skin treatment area; and wherein the coating has a thickness no greater than 0.5 mm. and includes the composition in an amount of from about 0.1 grams per 100 cm2 to about 3 grams per 100 cm.2.

Further embodiments of the above methods include the following:

1. The method of any other embodiment, wherein the spraying includes atomizing the composition with at least one of an atomizing spray dispenser, an atomizing pump spray dispenser, a piston-style dispenser, and an aerosol dispenser.
2. The method of any other embodiment, further comprising leaving said composition on the skin treatment area to form a coating; wherein the coating has a thickness no greater than 0.5 mm.
3. The method of any other embodiment, further comprising leaving said composition on the skin treatment area to form a coating; wherein the coating has a mass of from about 0.1 grams per 100 cm2 to about 3 grams per 100 cm2.
4. The method of any other embodiment, wherein the solid particulate material comprises particulate zinc oxide.
5. The method of any other embodiment, wherein the particulate zinc oxide has an average particle size of from about 0.01 microns to about 10 microns.
6. The method of any other embodiment, wherein the composition further comprises from about 5 percent to about 30 percent by weight of a skin conditioning agent.
7. The method of any other embodiment, wherein the skin conditioning agent comprises at least one of cod liver oil and lanolin.
8. The method of any other embodiment, wherein the composition further comprises from about 1 percent to about 10 percent by weight of a viscosity-increasing material.
9. The method of any other embodiment, wherein the viscosity-increasing material comprises at least one of talc, paraffin wax, and microcrystalline wax.
10. The method of any other embodiment, wherein the composition comprises from about 20 percent to about 30 percent by weight of the solid particulate material, wherein the solid particulate material is zinc oxide, from about 30 percent to about 60 percent by weight of the fluid base material, wherein the fluid base material comprises mineral oil and cyclomethicone; from about 1 percent to about 16 percent by weight of lanolin; from about 1 percent to about 25 percent by weight of petrolatum; from about 1 percent to about 10 percent by weight of microcrystalline wax; and from about 5 percent to about 12 percent by weight of cod liver oil.
11. The method of any other embodiment, wherein the wound is a weeping wound or a pressure ulcer.
12. The method of any other embodiment, wherein the composition further comprises silver in a concentration of 10 parts per million to 3000 parts per million.
13. The method of any other embodiment, wherein the coating has a thickness no greater than 0.5 mm.
14. The method of any other embodiment, wherein the coating has a mass of from about 0.1 grams per 100 cm2 to about 3 grams per 100 cm2.
15. The method of any other embodiment, wherein the particulate zinc oxide has an average particle from about 0.01 microns to about 1 micron.
16. The method of any other embodiment, wherein the composition further comprises one or more member selected from the group consisting of talc, lanolin, cod liver oil, petrolatum, paraffin wax and microcrystalline wax.
17. The method of any other embodiment, wherein the fluid base material comprises at least one of cyclomethicone and dimethicone.
18. The method of any other embodiment, wherein the solid material is selected from the group consisting of talc, calamine and kaolin.
19. The method of any other embodiment, wherein the composition further comprises at least one of a fragrance, a dye, a preservative, and an emollient.
20. The method of any other embodiment, further comprising: determining that the skin treatment area is at risk for exposure to ultraviolet radiation; and wherein the composition comprises from about 10 percent to about 30 percent by weight of hydrophobized zinc oxide and from about 20 percent to about 90 percent by weight of a fluid base material selected from the group consisting of mineral oil, silicone oil, plant-based oil and mixtures thereof.

Compositions described herein are effective in combating bacterial and fungal infections even without an additional antibacterial or antifungal ingredient since they are hygroscopic and allow diffusion of moisture across the coating to help keep the skin area dry. Possible bacteria that can be killed include: Clostridium difficile, Vancomycin Resistant Enterococcus faecalis (VRE), Methicillin Resistant Staphylococcus aureus (MRSA), Acinetobacter baumanii, Escherichia coli, Aspergillus niger, Escherichia coli, Bacillus atrophaeus, Mycobacterium bovis, Chlamydia trachomatis, Clostridium perfringens, Pseudomonas aeruginosa, Enterococcus hirae, Salmonella typhi, Candida albicans, parapsilasis, Aspergillus fumigaus Trichophyton mentagrophytes, and combinations thereof.

Various changes and modifications to the described embodiments described herein will be apparent to those skilled in the art, and such changes and modifications can be made without departing from the spirit and scope of the invention and without diminishing its intended advantages. Additionally, while the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected.

What is claimed is:

1. A method comprising:
identifying a skin treatment area having a wound which produces exudate;
determining that the skin treatment area is at risk for at least one of skin maceration, friction and shearing; and.
based at least in part on the determining, applying a composition to the skin treatment area, the composition comprising:
from about 1 percent to about 40 percent by weight of a solid particulate material;
from about 20 percent to about 99 percent by weight of a fluid base material comprising at least one material selected from the group consisting of mineral oil, silicone oil, plant-based oil; and
from about 0 percent to about 30 percent by weight of water;
wherein the applying includes spraying the composition such that the sprayed composition contacts the skin treatment area.

2. The method according to claim 1, wherein the spraying includes atomizing the composition with at least one of an atomizing spray dispenser, an atomizing pump spray dispenser, a piston-style dispenser, and an aerosol dispenser.

3. The method according to claim 1, further comprising leaving said composition on the skin treatment area to form a coating; wherein the coating has a thickness no greater than 0.5 mm.

4. The method according to claim 1, further comprising leaving said composition on the skin treatment area to form a coating; wherein the coating has a mass of from about 0.1 grams per 100 cm² to about 3 grams per 100 cm².

5. The method according to claim 1, wherein the solid particulate material comprises particulate zinc oxide.

6. The method according to claim 5, wherein the particulate zinc oxide has an average particle size of from about 0.01 microns to about 10 microns.

7. The method according to claim 1, wherein the composition further comprises from about 5 percent to about 30 percent by weight of a skin conditioning agent.

8. The method according to claim 7 wherein the skin conditioning agent comprises at least one of cod liver oil and lanolin.

9. The method according to claim 1, wherein the composition further comprises from about 1 percent to about 10 percent by weight of a viscosity-increasing material.

10. The method according to claim 9, wherein the viscosity-increasing material comprises at least one of talc, paraffin wax, and microcrystalline wax.

11. The method according to claim 1, wherein the composition comprises:
from about 20 percent to about 30 percent by weight of the solid particulate material, wherein the solid particulate material is zinc oxide,
from about 30 percent to about 60 percent by weight of the fluid base material, wherein the fluid base material comprises mineral oil and cyclomethicone;
from about 1 percent to about 16 percent by weight of lanolin;
from about 1 percent to about 25 percent by weight of petrolatum;
from about 1 percent to about 10 percent by weight of microcrystalline wax; and
from about 5 percent to about 12 percent by weight of cod liver oil.

12. The method according to claim 1, wherein the wound is a weeping wound or a pressure ulcer.

13. The method according to claim 1, wherein the composition further comprises silver in a concentration of 10 parts per million to 3000 parts per million.

14. A method comprising:
identifying a skin treatment area having a wound or rash;
determining that the skin treatment area would benefit from a covering that both al lows oxygen diffusion and provides a selective barrier to the passage of liquid;
providing a treatment system comprising a dispenser and a treatment composition;
wherein the dispenser comprises a container and an atomizing spray delivery mechanism affixed to the container; and wherein the treatment composition is positioned in the container;
passing the composition through the atomizing spray delivery mechanism to atomize the composition and to propel the atomized composition toward the skin treatment area; and
leaving said composition on the skin treatment area to form a coating;
wherein the treatment composition comprises from about 1 percent to about 40 percent by weight of a solid particulate material, from about 20 percent to about 99 percent by weight of a fluid base material selected from the group consisting of mineral oil, silicone oil, plant-based oil and mixtures thereof, and from 0 percent to about 30 percent water by weight; and
wherein the composition has a viscosity of from about 1 to about 1000 centipoise.

15. The method according to claim 14, wherein the coating has a thickness no greater than 0.5 mm.

16. The method according to claim 14, wherein the coating has a mass of from about 0.1 grams per 100 cm² to about 3 grams per 100 cm².

17. The method according to claim 14, wherein the solid material comprises particulate zinc oxide.

18. The method according to claim 17, wherein the particulate zinc oxide has an average particle from about 0.01 microns to about 1 micron.

19. The method according to claim 17, wherein the composition further comprises one or more member selected from the group consisting of talc, lanolin, cod liver oil, petrolatum, paraffin wax and microcrystalline wax.

20. The method according to claim 14, wherein the fluid base material comprises at least one of cyclomethicone and dimethicone.

21. The method according to claim 14, wherein the solid material is selected from the group consisting of talc, calamine and kaolin.

22. The method according to claim 14, wherein the composition further comprises at least one of a fragrance, a dye, a preservative, and an emollient.

23. The method according to claim 14, further comprising: determining that the skin treatment area is at risk for exposure to ultraviolet radiation; and wherein the composition comprises from about 10 percent to about 30 percent by weight of hydrophobized zinc oxide and from about 20 percent to about 90 percent by weight of a fluid base material selected from the group consisting of mineral oil, silicone oil, plant-based oil and mixtures thereof.

24. A method comprising:
identifying a skin treatment area that is in need of antimicrobial treatment; and
responding to the need by applying to the skin treatment area a composition comprising from about 1 percent to about 40 percent by weight of a solid particulate material, from about 20 percent to about 99 percent by weight of a fluid base material selected from the group consisting of mineral oil, silicone oil, plant-based oil and mixtures thereof, and from 0 percent to about 30 percent water by weight;
wherein the applying includes passing the composition through an atomizing spray delivery mechanism and into contact with the skin treatment area to form a coating on the skin treatment area;
wherein the coating has a thickness no greater than 0.5 mm and includes the composition in an amount of from about 0.1 grams per 100 cm2 to about 3 grams per 100 cm2.

25. The method according to claim 24, wherein the solid particulate material comprises particulate zinc oxide.

26. The method according to claim 24, wherein the composition further comprises silver in a concentration of 10 parts per million to 3000 parts per million.
